(19) United States
(12) Patent Application Publication
(10) Pub. No.: US 2018/0008325 A1
(43) Pub. Date: Jan. 11, 2018

(54) ADHESION ENHANCED CEMENT COATED INTERMEDULLARY NAIL

(71) Applicant: Rahul Vaidya, Ann Arbor, MI (US)

(72) Inventor: Rahul Vaidya, Ann Arbor, MI (US)

(21) Appl. No.: 15/203,794

(22) Filed: Jul. 7, 2016

(51) Int. Cl. A61B 17/72; A61L 31/18; A61L 31/16; A61L 31/08; A61B 17/00

(52) U.S. Cl. CPC A61B 17/72; A61L 31/16; A61L 31/088; A61L 31/18; A61B 2090/3966; A61B 2017/00893; A61L 2430/02

(57) ABSTRACT

A medication impregnated bone cement (MIBC) coated intramedullary (IM) nail for fixation of a long bone fracture comprising an IM nail base and medication impregnated bone cement. The bone cement encapsulates at least a portion of the IM nail base and forms an interface between the adjacent surfaces of the IM nail base and the bone cement. The interface between the encapsulating bone cement and the encapsulated IM nail base being enhanced to increase the adhesion of the encapsulating bone cement to the encapsulated IM nail base. The increase in adhesion being sufficient to ensure that the encapsulating bone cement remains adhered to the encapsulated IM nail base when the medication impregnated bone cement coated intramedullary nail is removed from the long bone.

### FIELD OF THE INVENTION

The present invention relates generally to intermedullary nails for fixation of long bone fractures and more specifically to medication impregnated bone cement (MIBC) coated intramedullary (IM) nails. Most specifically the present invention relates to medication impregnated bone cement (MIBC) coated intramedullary (IM) nails having an adhesion enhanced interface between the MIBC and the IM nail base.

### BACKGROUND OF THE INVENTION

Rod or nail placement in the medullary cavity for securing bone fractures is a common practice in orthopedic surgery. Use of the rod is known to inherently produce better healing in more extreme fractures than other procedures in which the rod is omitted. Spanning the fracture zone, the rod imposes a rigidity to the fracture area that could otherwise be difficult to maintain during the prolonged period of mending. When left permanently in place, the rod reinforces the bone and reduces its susceptibility to refracture.

The interlocking intramedullary nail has been widely used in the treatment of long bone fractures in recent years. However, patients who suffered from infection after nailing are hard to deal with. The current management of this kind of infection consists of two main objectives, one of which is infection control, which usually is achieved by nail removal with debridement, lavage of the medullary canal and local delivery of antibiotics. Unfortunately, removal of the intramedullary nail causes a high risk of non-union or additional fractures, challenging surgeons to prevent such fractures with few guidelines for how this can be done. Generally, external fixation is substituted for the removed IM nail.

To fight the infection, antibiotic-impregnated beads have been used to fill the dead space and deliver high concentration of specific antibiotics to the infected sites simultaneously, but some defects limit the application of antibiotic beads. Specifically, filling dead space incompletely, being hard to take out, and a short period of implantation time. These beads have been replaced by antibiotic spacers, and antibiotic PMMA-coated guide rods. While these methods address the problem of infection, all of these methods are temporary and provide no significant stability to the fracture, except for the antibiotic PMMA-coated guide rods, which provide limited stability to axial and bending forces but no rotational stability. Essentially, none of these methods results in stability that is comparable to a locked intramedullary nail, and none is likely to consistently achieve bony union without a subsequent procedure. For this reason, surgeons have started using medication impregnated bone cement (MIBC) coated IM nails to treat an infected long bone fractures.

U.S. Pat. No. 8,609,003 to Rahul Vaidya discloses a kit and method for forming medication impregnated bone cement (MIBC) intramedullary (IM) nails. The kit for forming medication impregnated bone cement (MIBC) intramedullary (IM) nails includes a molded tube including an attachment means at one end to allow for attachment of the molded tube to a conventional bone cement gun, the other end of the molded tube is adapted to allow for insertion of a IM nail thereinto. This allows for ease of creation of an medication impregnated bone cement coated intramedullary nail.

In use such an MIBC coated IM nail would be inserted into a fractured long bone and later removed once the bone has healed. Unfortunately, sometimes when the MIBC coated IM nail is removed from the patient, the IM nail base detaches from the MIBC coating and the coating remains within the IM canal. This seriously complicates the patient's healing process as additional procedures are required to extract the MIBC from the IM canal.

Thus there is needed in the art an MIBC coated IM nail with increased adhesion of the encapsulating bone cement to the encapsulated IM nail base.

### SUMMARY OF THE INVENTION

The present invention relates to a medication impregnated bone cement (MIBC) coated intramedullary (IM) nail for fixation of a long bone fracture comprising an IM nail base and medication impregnated bone cement. The bone cement encapsulates at least a portion of the IM nail base and forms an interface between the adjacent surfaces of the IM nail base and the bone cement. The interface between the encapsulating bone cement and the encapsulated IM nail base being enhanced to increase the adhesion of the encapsulating bone cement to the encapsulated IM nail base. The increase in adhesion being sufficient to ensure that the encapsulating bone cement remains adhered to the encapsulated IM nail base when the medication impregnated bone cement coated intramedullary nail is removed from the long bone.

The enhancement to increase the adhesion of the encapsulating bone cement to the encapsulated IM nail base may be created by a physical enhancement of the interface. The physical enhancement of the interface may comprise indentations on the surface of the IM nail base adjacent to the bone cement, the bone cement filling the indentations in the surface of the IM nail base. Alternatively, the physical enhancement of the interface may comprise protrusions projecting from the surface of the IM nail base adjacent to the bone cement, the protrusions projecting into the bone cement. Further, the physical enhancement of the interface may comprise both indentations on the surface of the IM nail base adjacent to the bone cement and protrusions projecting from the surface of the IM nail base. Alternatively, the physical enhancement of the interface may comprise an increased surface roughness of the IM nail base.

The enhancement to increase the adhesion of the encapsulating bone cement to the encapsulated IM nail base may alternatively be created by a chemical enhancement of the interface. The chemical enhancement of the interface may comprise formation of a chemical bond between the bone cement and the IM nail base. The formation of a chemical bond between the bone cement and the IM nail base may comprise deposition of an adhesive layer on the surface of the IM nail base to which the bone cement bonds. The adhesive layer may comprise a curing agent for the bone cement or a one part bio-compatible epoxy.

The formation of a chemical bond between the bone cement and the IM nail base may alternatively comprise an interface layer between the bone cement and the IM nail base, the interface layer being deposited on the IM nail base, the interface layer bonding more strongly with both of the bone cement and the material of the IM nail base than the bone cement bonds with the material of the IM nail base. The interface layer may comprise a layer of consisting of titanium dioxide (TiO₂) or aluminum oxide (Al₂O₃) deposited on the surface of the IM nail base. The interface layer may alternatively comprise a thin-film PMMA or CPC precoating deposited on the surface of the IM nail base to which the bone cement cross-links as it polymerizes.

The formation of a chemical bond between the bone cement and the IM nail base may also comprise a direct chemical bond between the bone cement and the surface of the IM nail base. The IM nail base may comprise a polymer which cross-links or copolymerizes with the bone cement as it polymerizes.

The IM nail base may comprise one or more bio-compatible materials selected from the group consisting of: metals, polymers, reinforced polymers, polymers with radiopaque additives and composite metal/polymer materials and combinations thereof. The IM nail base may comprise a PMMA or CPC polymer with embedded carbon fibers and a radiopaque contrast agent. The radiopaque contrast agent may comprise zirconium dioxide (ZrO₂) or barium sulphate (BaSO₄). The MIBC may be impregnated with one or more medications selected from the group consisting of antibiotics, antifungals, antineoplastics, and recombinant human bone morphogenetic protein.

### BRIEF DESCRIPTION OF THE FIGURES

FIG. 1 depicts a typical prior art MIBC coated IM nail with hardened MIBC formed about the IM nail base;

FIG. 2 depicts the prior art interface between the hardened MIBC and the IM nail base;

FIG. 3A depicts the interface between the hardened MIBC and the IM nail base and further shows indentations in the surface of the IM nail base;

FIG. 3B depicts a plate type IM nail base 2 having indentations that extend completely therethrough;

FIG. 4 depicts the interface between the hardened MIBC and the IM nail base and further shows protrusions from the surface of the IM nail base;

FIG. 5 depicts the enhanced interface between the hardened MIBC and the IM nail base which is caused by an increase in the surface roughness of the IM nail base; and

FIG. 6 depicts the chemically enhanced interface between the hardened MIBC and the IM nail base.

### DETAILED DESCRIPTION OF THE INVENTION

The present invention relates to improved medication impregnated bone cement (MIBC) coated intramedullary (IM) nails. One type of well known bone cement is Polymethyl-methacrylate (PMMA), PMMA is widely used for implant fixation in various Orthopaedic and trauma surgery. In reality, “cement” is a misnomer because the word cement is used to describe a substance that bonds two things together. However, PMMA acts as a space-filler that creates a tight space which holds the implant against the bone and thus acts as a grout. Bone cements have no intrinsic adhesive properties, but they rely instead on close mechanical interlock between the irregular bone surface and the prosthesis.

As detailed in U.S. Pat. No. 8,609,003 issued to Rahul Vaidya (the disclosure of which is hereby incorporated by reference), a medication impregnated bone cement (MIBC) coated intramedullary (IM) nail is created by insertion of a base IM nail into a mold and pumping a premixed liquid of medication impregnated bone cement into the mold. One type of bone cement, known as PMMA bone cement, is an acrylic polymer that is formed by mixing two sterile components: a liquid MMA monomer and a powered MMA-styrene co-polymer. When the two components are mixed, the liquid monomer polymerizes around the pre polymerized powder particles to form hardened PMMA. Bone cement has proven particularly useful because active substances, can be added to the powder component. This makes bone cement a modern drug delivery system that delivers the required drugs directly to the surgical site. Various antibiotics have been successfully mixed and used with bone cements like Gentamycin, Tobramycin, Erythromycin, Cefuroxime, Vancomycin, Colistin etc. The basic requirement, being that the mixable antibiotic should be heat resistant and should last for a relatively long duration of time.

Other types of medications useful in the MIBC of the present invention are antifungal drugs (eg. fluconazole and amphotericin B), antineoplastics (e.g. andriamycin and cisplatin), and recombinant human bone morphogenetic protein,

Other types of bone cement may also be used to coat the IM nail base. One such material are the calcium phosphate bone cements. Calcium phosphate cements (CPCs) are self-setting bioactive materials with unique properties for bone regeneration applications. CPCs can be used for the replacement of damaged bone. They are cementing systems consisting of powder and liquid phases. The mixing odf the phases causes a chemical reaction accompanied by setting and then hardening. CPCs combine features such as osteo-conductivity, the ability to maintain vital functions of osteogenic cells, biocompatibility, the lack of negative reactions from the body, and its fomability By controlling the phase composition it becomes possible to regulate the rate of biodegradation (dissolving in the body) of CPCs, allowing for the applicability of the material in bone tissue regeneration, so called tissue engineering. CPCs are without the drawbacks of polymer cements based on polymethyl-methacrylate caused by toxic degradation products. The weakly exothermic polymerization reactions of CPCs and their low shrinkage upon setting are very positive qualities of these cements. CPC cements can be durable and dense or porous. Their porosity is an important factor in their ability to deliver osteogenic cells and impregnated medications.

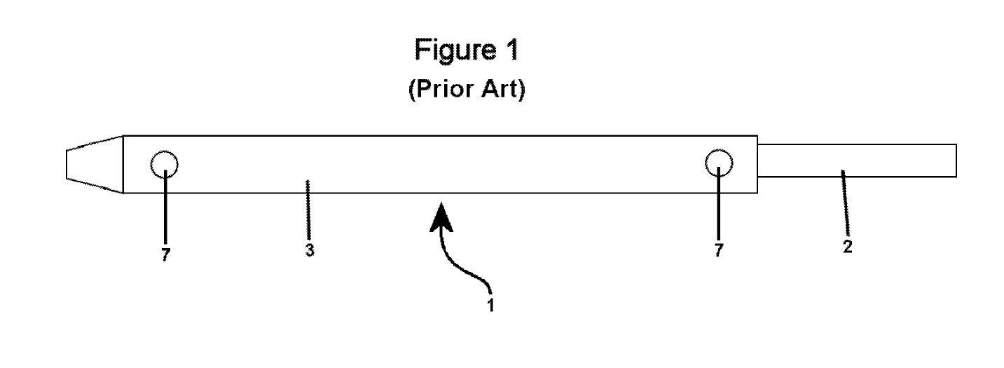

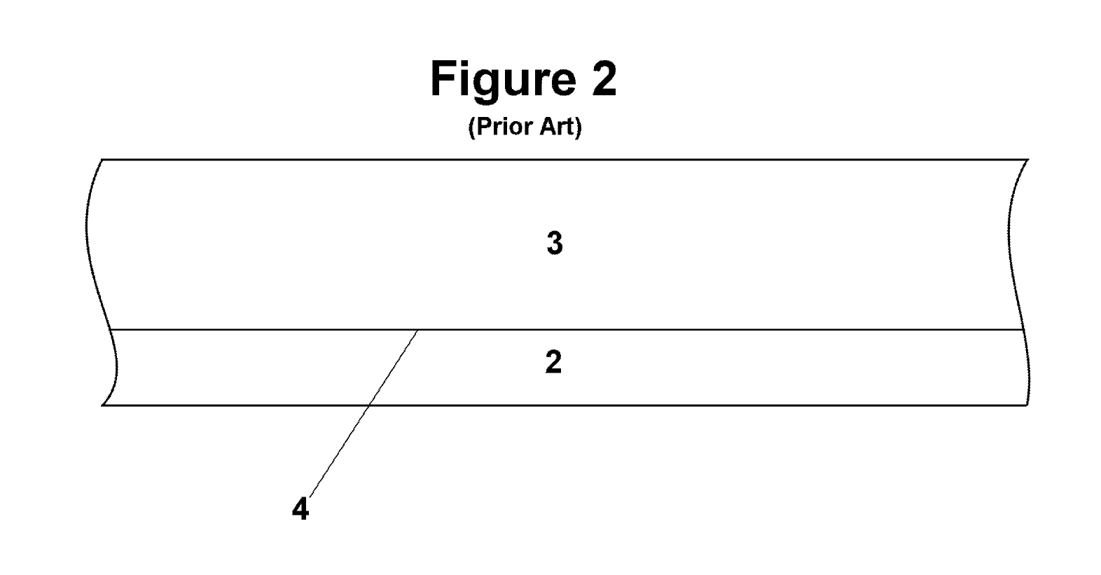

When medication impregnated bone cement sets/hardens it forms an encapsulating coating around the IM nail base. FIG. 1 depicts a typical (prior art) MIBC coated IM nail 1 with hardened MIBC 3 formed about the base IM nail 2 and including holes 7 to accommodate fastening means such as locking screws. FIG. 2 depicts the prior art interface 4 between the hardened MIBC 3 and the base IM nail 2. This interface 4 is generally smooth and this smoothness can provide inadequate adhesion between the base IM nail 2 and the MIBC 3. This can be especially true when the MIBC coated IM nail 1 is removed from the patient's bone after union and healing have occurred. If the MIBC separates from the base IM nail it may remain in the patient's IM canal, requiring additional surgical techniques to remove it. The can complicate and lengthen the healing process for the patient.

It is to this end that the present inventor has devised the improved MIBC coated IM nail of the instant invention. The present invention is an improved bone cement coated IM nail for fixation of a long bone fractures and includes an IM nail base 2 and bone cement 3 which encapsulates at least a portion of the IM nail base. Further, the interface between the encapsulating bone cement 3 and the encapsulated IM nail base 2 is enhanced to increase the adhesion of the encapsulating bone cement 3 to the encapsulated IM nail base 2, such that the increase in adhesion is sufficient to ensure that the encapsulating bone cement 3 remains adhered to the encapsulated IM nail base 2 when the cement coated IM nail is removed from the long bone.

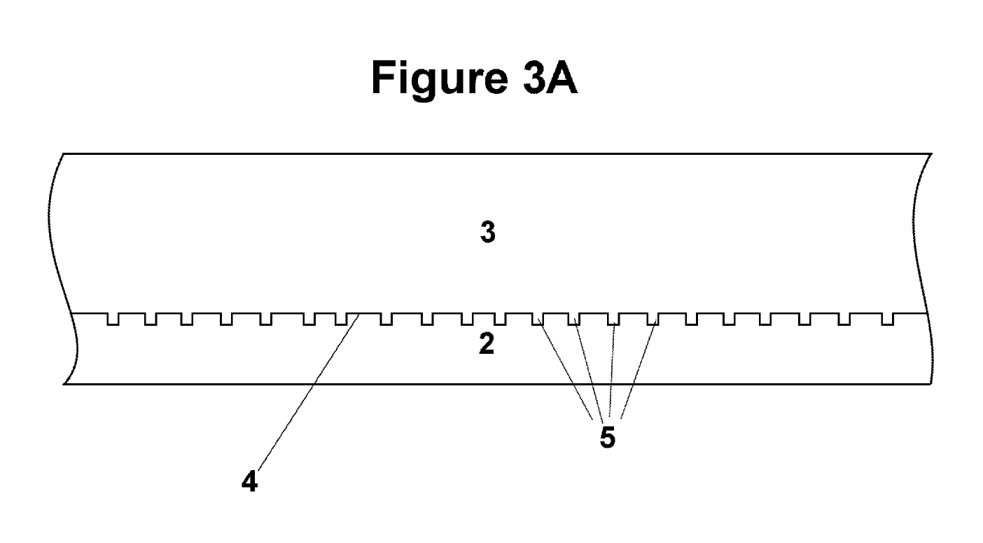

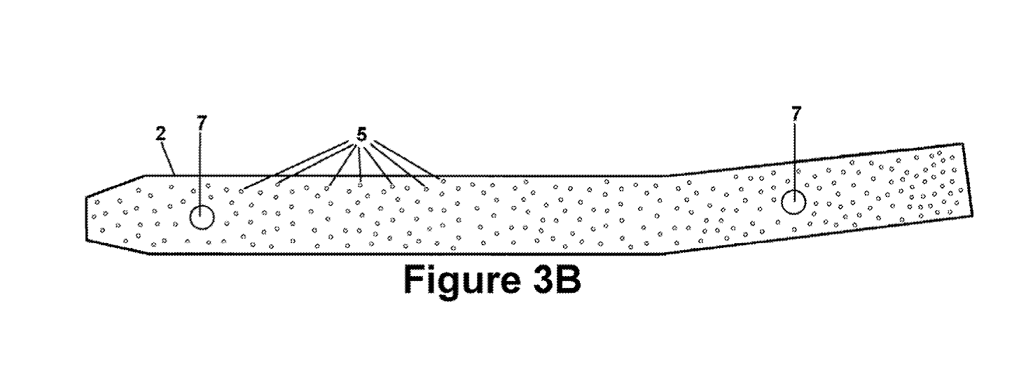

The increase in the adhesion strength between the bone cement and the base IM nail may be the result of physical enhancement of the IM nail base surface. Such a physical enhancement may come from creation of indentations in the surface of the base im nail. FIG. 3A depicts the interface 4 between the hardened MIBC 3 and the base IM nail 2 and further shows indentations 5 in the surface of the base IM nail 2. The indentations 5 increase the surface area of the interface 4 between the MIBC 3 and the base IM nail 2. The indentations 5 also allow for interpenetration of the MIBC 3 into the base IM nail 2, which in turn allows for increased adhesion as the coated im nail is removed from the patient's long bone. The indentations 5 may be deep or shallow as needed and may be angled into the base nail so as to increase the adhesion with respect to forces acting on the interface during the removal of the coated nail. It should also be noted that the indentations 5 may traverse the entire depth of the IM nail base such that the MIBC infiltrates all the way through the IM nail base. The indentations 5 may be placed and spaced evenly on the base IM nail surface or may be randomly spaced and placed. The indentations 5 may also be placed/distributed in a such a way as to enhance the adhesion in critical areas of the interface as needed or desirable. The indentations 5 may be uniform in size and shape across the surface of the base IM nail or may be different shapes and sizes as needed, or even random shapes and sizes.

FIG. 3B depicts a plate type IM nail base 2 having indentations 5 in the surface there which may extend completely therethrough. The IM nail base 2 also includes holes 7 to accommodate fastening means such as locking screws. As noted above when the indentations 5 extend completely through the entire depth of the IM nail base 2, the MIBC infiltrates all the way through the IM nail base 2, thereby allowing for enhanced adhesion of the MIBC to the IM nail base.

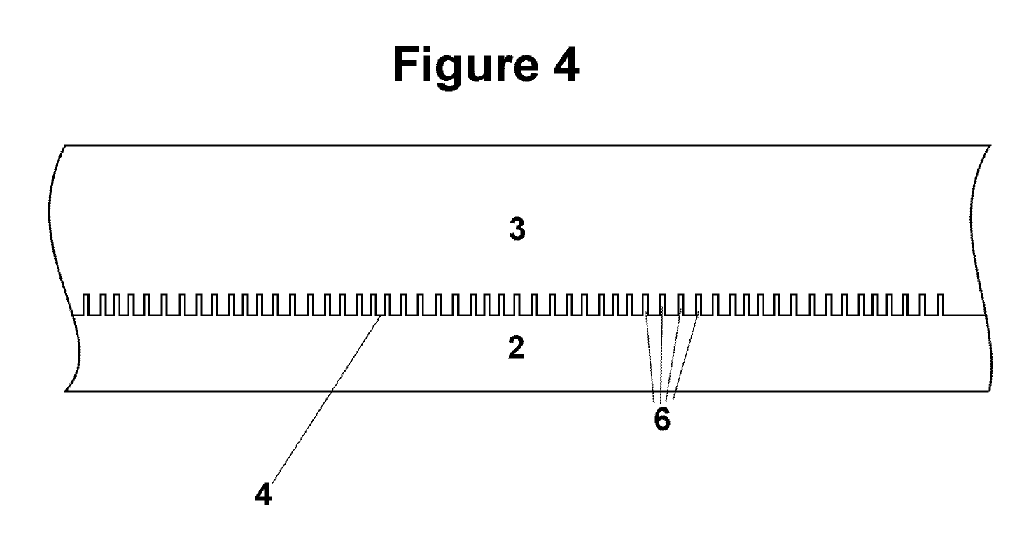

Another physical enhancement of the surface of the base IM nail may come from creation of protrusions from the surface of the base im nail. FIG. 4 depicts the interface 4 between the hardened MIBC 3 and the base IM nail 2 and further shows protrusions 6 from the surface of the base IM nail 2. The protrusions 6 increase the surface area of the interface 4 between the MIBC 3 and the base IM nail 2. The protrusions 6 also allow for interpenetration of the base IM nail 2 into the MIBC 3, which in turn allows for increased adhesion as the coated im nail is removed from the patient's long bone. The protrusions 6 long or short as needed and may be angled from the base nail so as to increase the adhesion with respect to forces acting on the interface during the removal of the coated nail. The protrusions 6 may be placed and spaced evenly on the base IM nail surface or may be randomly spaced and placed. The protrusions 6 may also be placed/distributed in a such a way as to enhance the adhesion in critical areas of the interface as needed or desirable. The protrusions 6 may be uniform in size and shape across the surface of the base IM nail or may be different shapes and sizes as needed, or even random shapes and sizes.

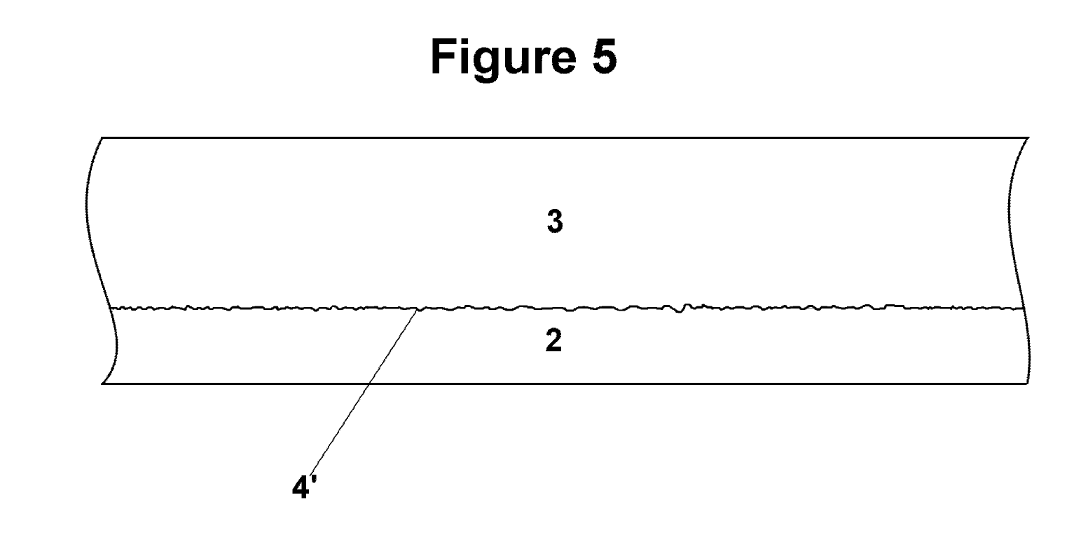

It should be noted that a combination of both indentations 5 and protrusions 6 may also be used if desirable to further enhance the adhesion of the encapsulating bone cement 3 to the encapsulated IM nail base 2. This combination of both indentations 5 and protrusions 6 is a macroscopic version of yet another physical enhancement of the surface of the base IM nail. This third example of physical enhancement of the surface of the base IM nail is an increase in the surface roughness of the surface of the base im nail. This increase in surface roughness can be thought of as a microscopic combination of both indentations 5 and protrusions 6 on the surface of the base IM nail. FIG. 5 depicts the enhanced interface 4′ between the hardened MIBC 3 and the base IM nail 2 which is caused by an increase in the surface roughness of the base IM nail.

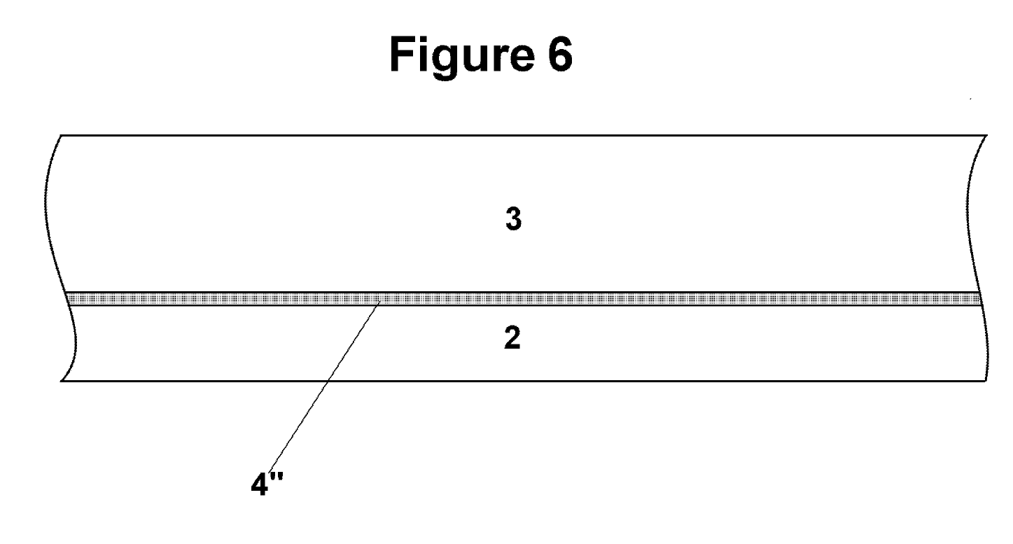

Another method of enhancing the adhesion between the encapsulating bone cement 3 and the encapsulated IM nail base 2 is by chemical enhancement. That is, by chemical enhancement of the bond between the IM nail base and the bone cement. FIG. 6 depicts the chemically enhanced interface 4″ between the hardened MIBC 3 and the base IM nail 2. The chemically enhance interface 4″ may include an adhesive layer on the base IM nail, an interface layer (placed between the bone cement and IM nail base) which bonds more strongly to each of the bone cement and the IM nail base than does the bone cement directly to the nail base, and a direct chemical bond between the bone cement and the IM nail base.

The adhesive layer is deposited on the IM nail base and may be any adhesive that is bio-compatible and bonds to both bone cement and the material of the IM nail base. Some examples of such an adhesive are a curing agent for the bone cement and a one part bio-compatible epoxy. The interface layer between the bone cement and the IM nail base is deposited on the IM nail base and bond more strongly with both of the bone cement and the material of the IM nail base than the bone cement bonds with the material of the IM nail base. The interface layer may act as a primer allowing the bone cement to adhere more strongly to the base IM nail. Such a primer layer may be a layer of titanium dioxide (TiO₂) or aluminum oxide (Al₂O₃). The primer may also be a thin-film PMMA precoating of the IM nail base to which the bone cement may cross-link as it polymerizes. Finally, the bone cement may be directly chemically bonded with the base IM nail. This can be accomplished by creation of a base IM nail formed from a polymer which reacts (cross-links or copolymerizes) with the bone cement as it polymerizes.

The base IM nail may be formed from bio-compatible metals, polymers, reinforced polymers, polymers with radio-opaque additives and composite metal/polymer materials and combinations thereof. One useful base IM nail may be a PMMA polymer with embedded carbon fibers and a radiopaque contrast agent. Commercially available cements use either zirconium dioxide (ZrO₂) or barium sulphate (BaSO₄) as the radiopaque contrast agent. Zirconium dioxide is one hundred times less soluble than barium sulphate and has less effect on the mechanical properties of the cement.

As described in U.S. Pat. No. 8,609,003 patent, instead of the base metal IM nail, the cement nail can be formed on other base/support elements. For example, the base may be a metal strip or plate with holes through the strip in the proper locations to allow for spacers to create locking screw holes in the molded cement nail. In addition to the locking screw holes, the strip or plate may include indentations that extent all the way through the strip or plate allowing MIBC to infiltrate completely therethrough. This same concept of indentations extending through the IM nail base can be applied to convention Another alternative may be metal wires, such as straight or curved wires, coils, flat mesh, or mesh cages. The metal wires act like reinforcing steel bar in concrete structural constructions. This wire structure is useful because the cement nail alone (without the support) is strong in compression, but is weak in tension. Thus the base provides added tension strength to the cement nail. While metal wire is described above, other materials may be used to form the support as long as it adds to the strength of the cement nail. If the base support is not adapted to be attached to an IM nail insertion handle, a proximal end stub of a conventional IM nail may be formed into the cement nail.

It is to be understood that the disclosure set forth herein is presented in the form of detailed embodiments described for the purpose of making a full and complete disclosure of the present invention, and that such details are not to be interpreted as limiting the true scope of this invention as set forth and defined in the appended claims.

I claim:

1. A medication impregnated bone cement (MIBC) coated intramedullary (IM) nail for fixation of a long bone fracture comprising:
an IM nail base; and
medication impregnated bone cement;
wherein said bone cement encapsulates at least a portion of said IM nail base and forms an interface between the adjacent surfaces of said IM nail base and said bone cement;
said interface between said encapsulating bone cement and said encapsulated IM nail base being enhanced to increase the adhesion of said encapsulating bone cement to said encapsulated IM nail base;
wherein said increase in adhesion is sufficient to ensure that said encapsulating bone cement remains adhered to said encapsulated IM nail base when said medication impregnated bone cement coated intramedullary nail is removed from said long bone.

2. The MIBC coated IM nail of claim 1, wherein said enhancement to increase the adhesion of said encapsulating bone cement to said encapsulated IM nail base is created by a physical enhancement of said interface.

3. The MIBC coated IM nail of claim 2, wherein said physical enhancement of said interface comprises indentations on said surface of said IM nail base adjacent to said bone cement, said bone cement filling said indentations in said surface of said IM nail base.

4. The MIBC coated IM nail of claim 2, wherein said physical enhancement of said interface comprises protrusions projecting from said surface of said IM nail base adjacent to said bone cement, said protrusions projecting into said bone cement.

5. The MIBC coated IM nail of claim 2, wherein said physical enhancement of said interface comprises both:
a) indentations on said surface of said IM nail base adjacent to said bone cement, said bone cement filling said indentations in said surface of said IM nail base; and
b) protrusions projecting from said surface of said IM nail base adjacent to said bone cement, said protrusions projecting into said bone cement.

6. The MIBC coated IM nail of claim 2, wherein said physical enhancement of said interface comprises an increased surface roughness of said IM nail base.

7. The MIBC coated IM nail of claim 1, wherein said enhancement to increase the adhesion of said encapsulating bone cement to said encapsulated IM nail base is created by a chemical enhancement of said interface.

8. The MIBC coated IM nail of claim 7, wherein said chemical enhancement of said interface comprises formation of a chemical bond between said bone cement and said IM nail base.

9. The MIBC coated IM nail of claim 8, wherein said formation of a chemical bond between said bone cement and said IM nail base comprises deposition of an adhesive layer on said surface of said IM nail base to which said bone cement bonds.

10. The MIBC coated IM nail of claim 9, wherein said adhesive layer comprises a curing agent for said bone cement.

11. The MIBC coated IM nail of claim 9, wherein said adhesive layer comprises a one part bio-compatible epoxy.

12. The MIBC coated IM nail of claim 8, wherein said formation of a chemical bond between said bone cement and said IM nail base comprises an interface layer between said bone cement and said IM nail base, said interface layer being deposited on the IM nail base, said interface layer bonding more strongly with both of said bone cement and the material of said IM nail base than said bone cement bonds with said material of said IM nail base.

13. The MIBC coated IM nail of claim 12, wherein said interface layer comprises a layer of consisting of titanium dioxide (TiO₂) or aluminum oxide (Al₂O₃) deposited on said surface of said IM nail base.

14. The MIBC coated IM nail of claim 12, wherein said interface layer comprises a thin-film PMMA or CPC precoating deposited on said surface of said IM nail base to which said bone cement cross-links as it polymerizes.

15. The MIBC coated IM nail of claim 8, wherein said formation of a chemical bond between said bone cement and said IM nail base comprises a direct chemical bond between said bone cement and said surface of said IM nail base.

16. The MIBC coated IM nail of claim 15, wherein said IM nail base comprises a polymer which cross-links or copolymerizes with said bone cement as it polymerizes.

17. The MIBC coated IM nail of claim 1, wherein said IM nail base comprises one or more bio-compatible materials selected from the group consisting of: metals, polymers, reinforced polymers, polymers with radiopaque additives and composite metal/polymer materials and combinations thereof.

18. The MIBC coated IM nail of claim 17, wherein said IM nail base comprises PMMA or CPC polymer with embedded carbon fibers and a radiopaque contrast agent.

19. The MIBC coated IM nail of claim 18, radiopaque contrast agent comprises zirconium dioxide (ZrO₂) or barium sulphate (BaSO₄).

20. The MIBC coated IM nail of claim 1, wherein said MIBC is impregnated with one or more medications selected from the group consisting of antibiotics, antifungals, antineoplastics, and recombinant human bone morphogenetic protein.
